(19) United States
(12) Patent Application Publication
(10) Pub. No.: US 2003/0192535 A1
(43) Pub. Date: Oct. 16, 2003

(54) Inhaler

(75) Inventors: Soren Christrup, Struer (DK); Anders Geert-Jensen, Hojbjerg (DK); Mikael Jorgensen, Arhus C (DK); Jorgen Rasmussen, Struer (DK); Hugo Dines Schmidt, Arhus C (DK)

Correspondence Address: NIXON & VANDERHYE, PC, 1100 N GLEBE ROAD, 8TH FLOOR, ARLINGTON, VA, 22201-4714, US

(73) Assignee: AstraZeneca AB.

(21) Appl. No.: 10/438,057

(22) Filed: May 15, 2003

Related U.S. Application Data
(63) Continuation of application No. 10/105,370, filed on Mar. 26, 2002, now abandoned.
Application No. 10/105,370 is a continuation of application No. 09/873,385, filed on Jun. 5, 2001, now abandoned.
Application No. 09/873,385 is a continuation of application No. 09/424,336, filed on Nov. 22, 1999, now abandoned.

(30) Foreign Application Priority Data
Sep. 24, 1999 (WO) PCT/SE99/01684

(51) Int. Cl. A61M 11/00

(52) U.S. Cl. 128/200.23

(57) ABSTRACT

An inhaler such as a breath-actuated inhaler for delivering medicament by inhalation, comprising a housing for receiving a canister of medicament actuatable to deliver a dose of medicament, a detector for detecting actuation of a received canister, a timer responsive to the detector to measure the elapsed time from actuation of the canister, and an indicator means responsive to the timer to indicate when a predetermined time has elapsed during which the user should maintain inhalation or delay exhalation. This allows the inhaler to provide an indication of how the user should breath to properly receive a dose of medicament. The indicator means may be further responsive to the timer to indicate when a second predetermined time has elapsed.

The present invention relates to an inhaler, such as a breath-actuated inhaler, for delivering medicament by inhalation.

Known inhalers receive a canister of medicament which is actuatable to deliver a dose of medicament. For various medicaments, the user is required to synchronise inhalation and exhalation with the actuation of the canister. Normally it is recommended that the user inhales for a given period of time after delivery of a dose. However, in practice this can be difficult for the user who will often breath incorrectly resulting in inhalation of an incorrect dose or in a dose not properly reaching the user's lungs or throat. This can be particularly a problem for the user in an emergency when it may be more vital than usual to deliver a dose properly.

It is known to provide a breath-actuated inhaler which includes an actuation mechanism operatable to actuate the canister upon inhalation by the user. This ensures that the dose of medicament delivered on actuation of the canister is supplied whilst the user is inhaling. However, there remains the problem of the user needing to synchronise the rest of his inhalation with the delivery of a dose, in particular as to how long the user should maintain inhalation or delay exhalation. This is particularly difficult in the case of a breath-actuated inhaler because the user does not always recognise what point during inhalation the breath-actuated mechanism actuates the cannister to deliver a dose of medicament.

The present invention is intended to assist the user in synchronising breathing with delivery of a dose.

According to the present invention there is provided an inhaler for delivering medicament by inhalation, comprising:

a housing for receiving a canister of medicament actuatable to deliver a dose of medicament;

a detector for detecting actuation of a received canister;

a timer responsive to the detector to measure the elapsed time from actuation of the canister; and

an indicator means responsive to the timer to indicate when a predetermined time has elapsed during which the user should maintain inhalation or delay exhalation.

As a result, the user is provided with an indication of the time for which he should maintain inhalation or delay exhalation which allows the user to fully synchronise his breathing with the delivery of a dose. This assists in assuring that the dose is properly inhaled by the user. The present invention is advantageous because it overcomes the difficulties for the user in determining when the actual actuation of the of the canister occurred, especially in a breath-actuated inhaler. As the inhaler detects inhalation and indicates when the predetermined time has elapsed relieves the user from this task and enables the user to breath correctly to ensure proper delivery of a dose.

Typically the period of time is from 0.2 to 15 seconds.

The indicator means may comprise means for indicating the period of time by visual indication, audible indication or by physically interacting with the user.

To provide a visual indication, the indicator means may be a display. The display may be arranged to alter the information displayed, such as a representation of the number of doses, after the predetermined time has elapsed.

Another manner of providing a visual indication is for the indicator means to comprise means for automatically re-setting the actuation mechanism, for example by moving buttons or other means for mechanically introducing energy into the inhaler.

The indicator means may be arranged to physically move the inhaler for example by vibrating the inhaler.

The indicator means may be arranged to generate an audible indication. A sound may be generated after the elapsed time or the generation of a sound of the entire predetermined elapsed time followed by change of or cessation in the sound.

Naturally, the different manners of indication may be combined.

Advantageously the indicator means is further responsive to the timer to indicate when a second predetermined time has elapsed. Thus, the first predetermined time may be a time during which the user should maintain inhalation and the second predetermined time may be a time during which the user should delay exhalation. This allows the inhaler to provide further information for assisting the user in correctly breathing when using the inhaler.

To allow a better understanding, an inhaler which embodies the present invention will now be described by way of non-limitative example with reference to the accompanying drawings, in which:

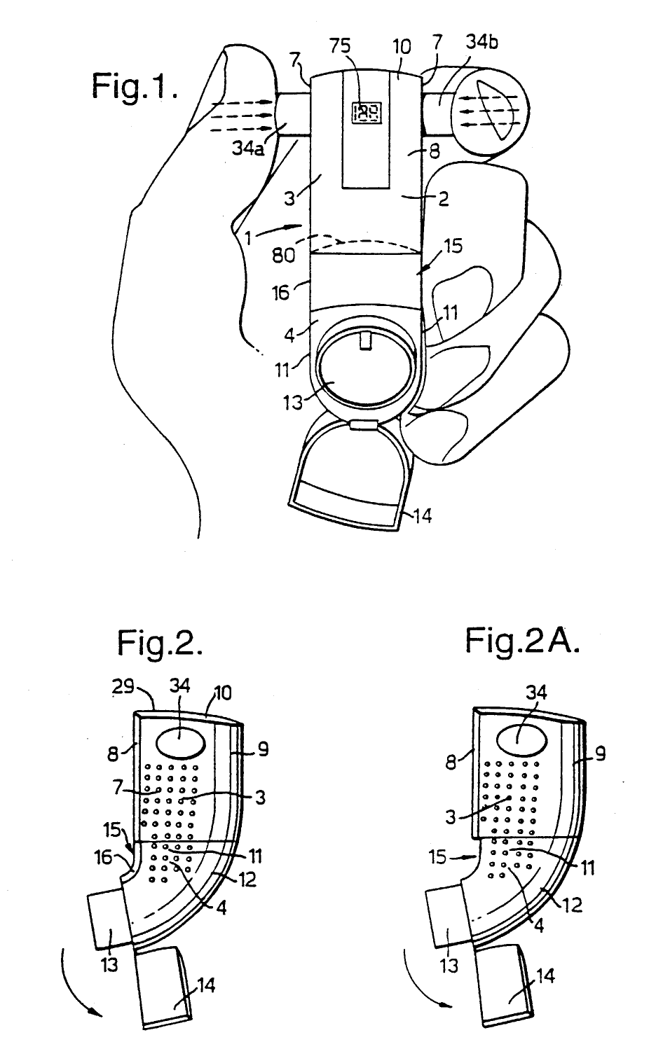

FIG. 1 is a front view of the inhaler held in a hand;

FIG. 2 is a side view of the inhaler;

FIG. 2A is a side view of the inhaler without a closure element fitted;

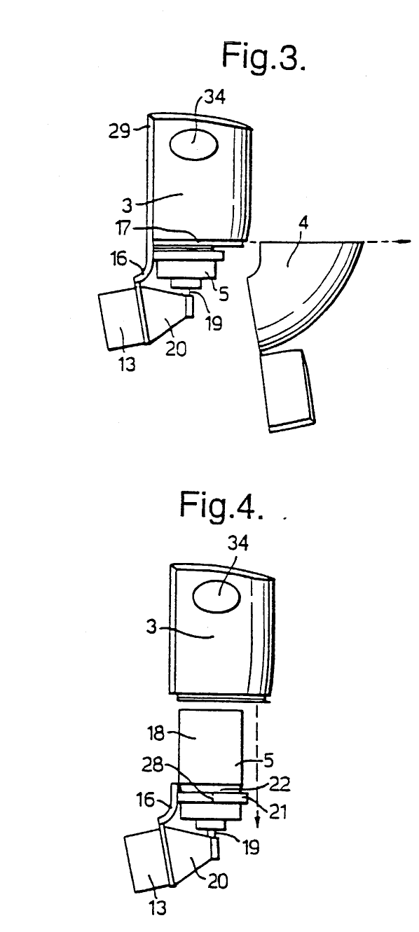

FIG. 3 is a side view of the inhaler with a lower housing portion being removed;

FIG. 4 is a side view of an upper housing portion of the inhaler with a canister being removed;

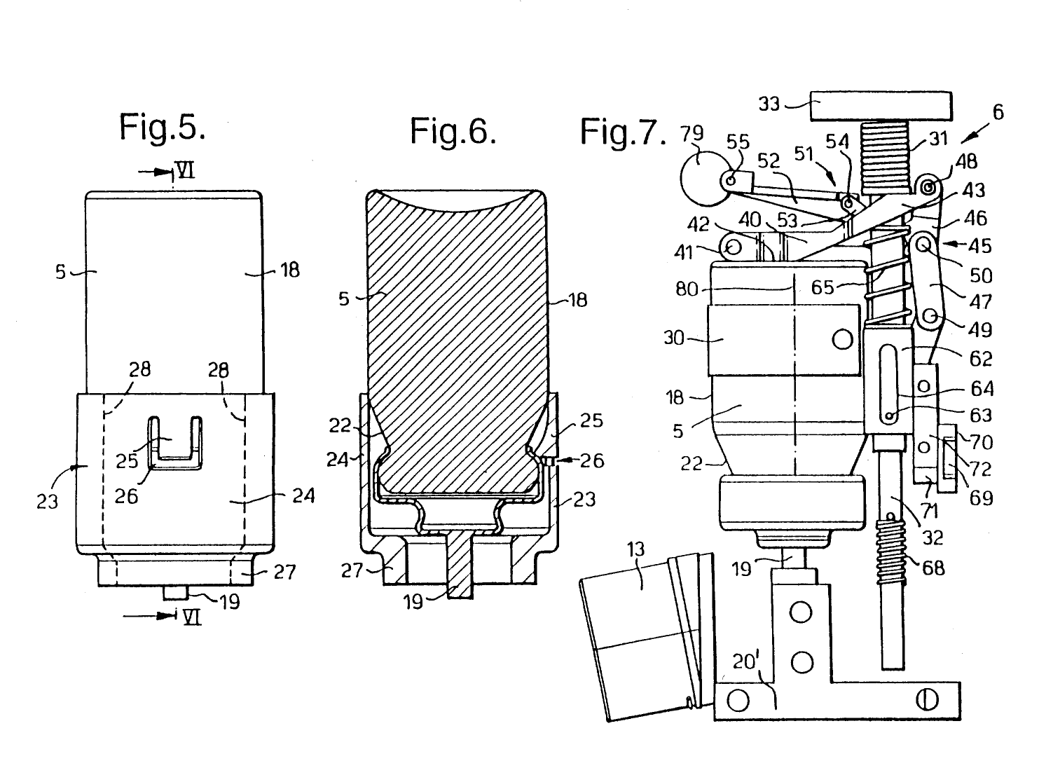

FIG. 5 is a side view of an alternative form of collar for connecting the closure element to the canister;

FIG. 6 is a cross-sectional view taken alone line VI-VI in FIG. 5;

FIG. 7 is a side view of the canister mounting arrangement and actuation mechanism;

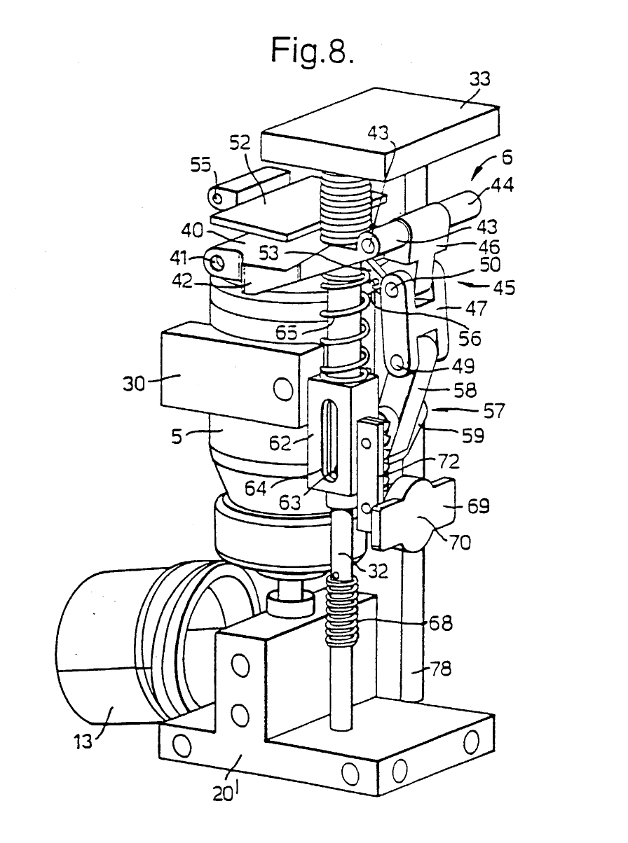

FIG. 8 is a view from the rear and side of the actuation mechanism;

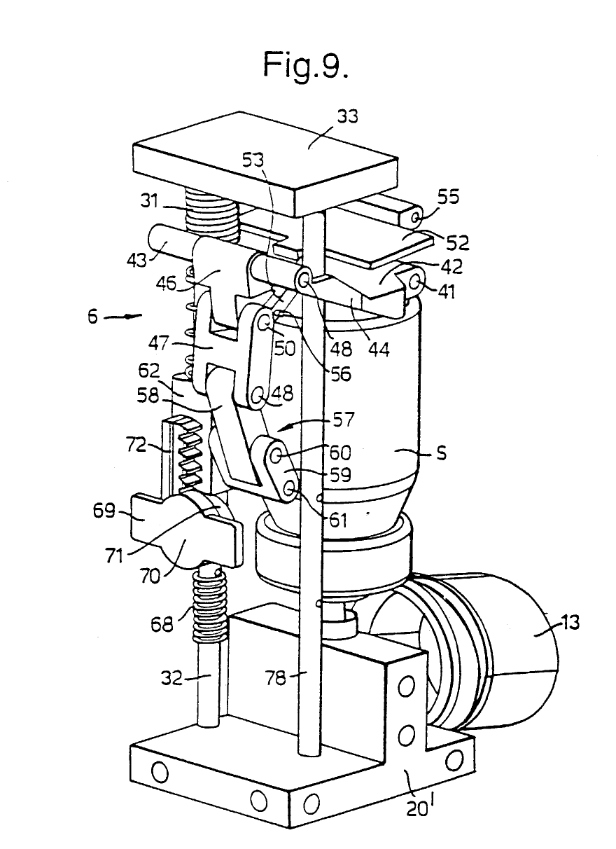

FIG. 9 is a view from the rear and the opposite side from FIG. 8 of the actuation mechanism;

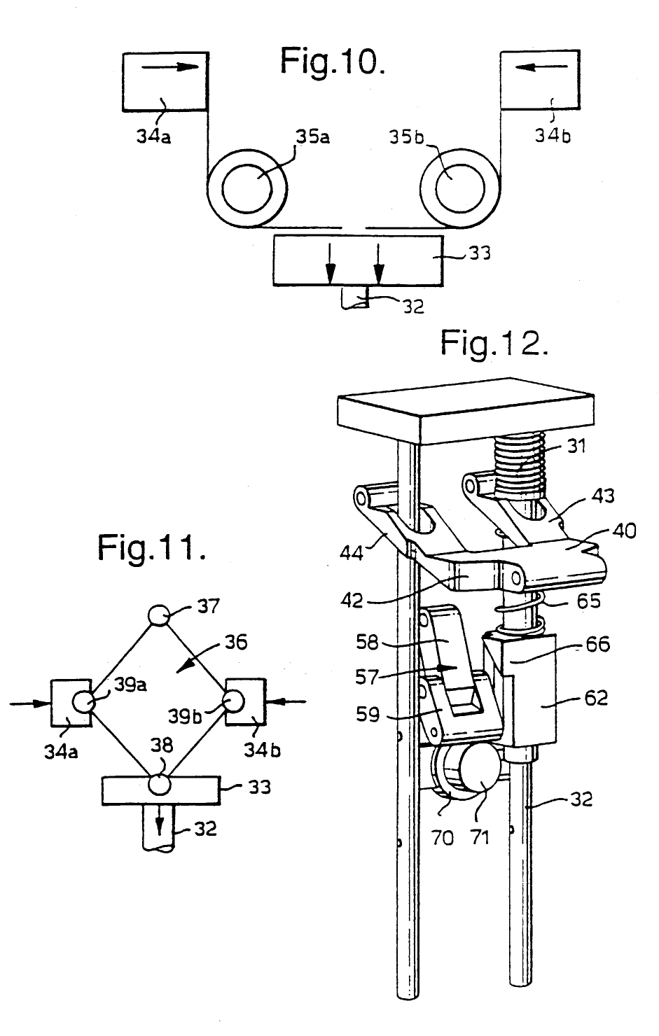

FIG. 10 is a front view of the arrangement for loading the actuation mechanism;

FIG. 11 is a side view of an alternative form of button arrangement for loading the actuation mechanism;

FIG. 12 is a view of certain parts of the actuation mechanism from the front and side;

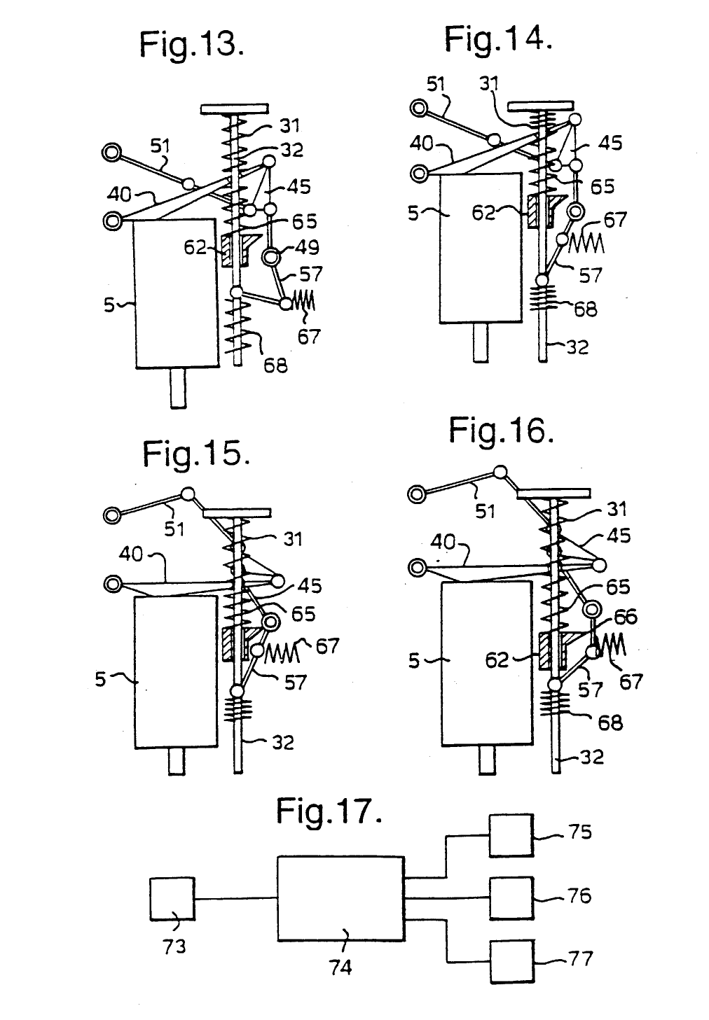

FIGS. 13 to 16 are schematic views of the actuation mechanism illustrating respective states over a complete cycle of operation; and

FIG. 17 is a view of the electronic timer circuit.

An inhaler 1 which embodies the present invention is illustrated in FIGS. 1 and 2, respectively showing the front view of the inhaler 1 held in a users hand and a side view of the inhaler.

The inhaler has a housing 2 comprising an upper housing portion 3 and a lower housing portion 4 which are coupled together. The upper and lower housing portions 3 and 4 have outer walls which are hollow to define a space accommodating a canister 5 of medicament and an actuation mechanism 6 operatable to actuate the canister 5 to deliver a dose of medicament.

The upper housing portion 3 has opposed side walls 7 joined by a flat front wall 8, a curved rear wall 9 and a top wall 10. The lower housing portion 3 has opposed side walls 11 fitting flush with the side walls 7 of the upper housing portion 3 and a curved rear wall 11 fitting flush with the rear wall 9 of the upper housing portion 3. The rear walls 11 and 9 together form a curved surface comfortably received in the palm of the user's hand as illustrated in FIG. 1. A mouthpiece 13 protrudes from the lower housing portion 4 and may be protected by a cap 14 hinged to the lower housing member 4 to be openable as illustrated in FIG. 2.

The front of the lower housing member 4 between the side walls 11 is open to define a vent in the outer surface of the housing 2 adjacent the mouthpiece 13 between the upper and lower housing portions 3 and 4. The vent 15 is closed by a closure element 16 fitting flush with the front wall 8 of the upper housing portion 3 to form part of the outer wall of the housing 2.

The upper and lower housing members are coupled by a coupling 17 allowing the lower housing member 4 to be slid off as illustrated in FIG. 3.

The canister 5 fits in the upper housing portion 3 and may be slidably removed for replacement as illustrated in FIG. 4.

The canister 5 comprises a generally cylindrical body 18 and a valve stem 19 which are compressible together to deliver a dose of medicament from the valve stem 19. The canister is of a known type including a metering chamber which captures a defined volume of medicament from the body 18 of the canister 5, which volume of medicament is delivered as a metered do from the valve stem 19 on compression of the valve stem 19 relative to the body 18. The valve stem 19 is weakly biassed outwardly to reset the canister 5 after compression for refilling the metering chamber. The valve stem 19 is received in a nozzle block 20 which is arranged to direct a dose of medicament delivered from the valve stem 19 out of the inhaler 1 through the mouthpiece 13.

The closure element 16 is connected to the canister 5 by a collar 21 fitted around a necked portion 22 of the canister body 18. The collar 21 is permanently fixed to the closure element 16 and may be integral therewith. The collar 21 is restrained by the necked portion 22 of the canister 5 such that the closure element 16 is removed and replaced together with the canister 5 as illustrated in FIG. 4. The canister 5 and collar 21 have a small degree of relative movement along the axis of the canister 5. This allows actuation of the canister by compression of the canister body 18 towards the valve stem 19 when the stem 19 is fixed relative to the inhaler 1 in the nozzle block 20 and the collar 21 is also fixed by the closure element 16 fitting as part of the housing 2 of the inhaler 1.

FIGS. 5 and 6 respectively illustrate a side view and cross-sectional view and alternative collar 23 for connecting the closure element 16 to the canister 5. The collar 23 includes a cylindrical portion 24 held on the necked portion 22 of the canister body 18 by a protrusion 25 formed in the cylindrical portion 24 by a U-shaped cut-out 26. The cylindrical portion 24 has an extension 27 extending beyond the end of the canister body 18 to protect the valve stem 19. The extension 27 is of reduced diameter relative to the remainder of the cylindrical portion 24 of the collar 23.

The collars 21 and 23 are both formed with a weak portion constituted by two rupture lines 28 disposed on opposite sides of the collar 21 or 23 and arranged to be broken preferentially to the remainder of the collar 21 or 23 on application of a force to separate the closure element 16 from the canister 5. After the rupture lines 28 have been broken or at least deformed to enable removal of the canister 5, it is impossible to connect the collar 21 or 23 to a different canister.

The outer surface of the closure element 16 carries an indication of the type of medicament in the canister 5 to which the closure element 16 is connected. The indication may be printed information, an embossed or indented pattern or the colour of the closure element 16.

An inlet opening 29 is formed in the upper housing portion 3, in particular in its top wall 10 and front wall S. The outer walls of the housing defined by the upper and lower portions 3 and 4 and the closure element 16 seal together to define a closed space which constitutes an air flow path extending from the mouthpiece 13 through the housing 2 to the inlet opening 29. Inhalation at the mouthpiece 13 draws air in through the inlet opening 29 through that air flow path around the canister 5 and actuation mechanism 6 encased in the housing 2. The actuation mechanism 6 (described in detail below) has a trigger disposed in the upper housing portion 4 which, in response to a flow through the air flow path, triggers the actuation mechanism 6 to actuate the canister 5.

If a canister without a closure element connected thereto is inserted into the housing 2, then the vent 15 will remain open as illustrated in FIG. 2A. Consequently, when a user inhales at the mouthpiece 13, the flow resistance through the vent 15 will be much lower than the flow resistance through the remainder of the air flow path above the vent 15 from the inlet opening 9. Accordingly, the vent 15 will vent most of the flow through the mouthpiece, thereby reducing the flow in the remainder of the air flow path in the upper housing portion through. The positioning of the vent 15 in the air flow path inside the housing 2 between the mouthpiece 13 and the trigger reduces the air flow across the trigger. The vent 15 is positioned and dimensioned such that the flow at the trigger is reduced below the threshold needed to operate the trigger and therefore prevents operation of the actuation mechanism 6. To assist in assuring that the vent 15 sufficiently vents the flow, the vent 15 is provided with a larger opening area and hence a lower flow resistance than the inlet opening 29. The vent 15 is dimensioned so that the actuation mechanism is not operated on a flow through the mouthpiece 13 at a level above the maximum expected inhalation, for example at an inhalation of at least eight times a standard inhalation flow rate. The triggering mechanism for the actuation mechanism 6 is designed taking into account the flow generated by a standard inhalation selected by the designer.

The actuation mechanism 6 for actuating the canister 5 to deliver a dose of medicament is illustrated in FIGS. 7 to 9. The elements illustrated in FIGS. 7 to 9 are accommodated in the housing 2 but are illustrated separately for clarity. The canister 5 is held with its valve stem 19 in a nozzle block 20′ connected to the mouthpiece 13, both fixed relative to the lower housing portion 4. A nozzle block 20′ has a slightly different structural form from the nozzle block 20 illustrated in FIGS. 3 and 4 but performs the same function. The body 18 of the canister 5 is supported by a guide block 30 fixed to the upper housing portion 3 and having a curved inner surface engaging the cylindrical surface of the canister body 18 to allow axial movement of the canister body 18 within the housing 2. The actuation mechanism 6 operates to compress the canister body 18 relative to the valve stem 19 held in the nozzle block 20 to deliver a dose of medicament.

The structure of the actuation mechanism 6 is as follows.

The actuation mechanism 6 includes a pre-loading mechanism for loading a resilient loading element in the form of a coiled loading spring 31. The pre-loading mechanism includes the loading member constituted by a shaft 32 encircled by the coils of the loading spring 31. The shaft extends and is movable in a direction parallel to the cylindrical axis 80 of the canister body 18. The loading member shaft 32 has an enlarged head 33.

As illustrated in FIG. 1, two buttons 34a and 34b, constituting contact members to be manually depressed are mounted opposite one another in the side walls 7 of the upper housing portion 3 on either side of the axis 80 of the canister 5 held in the housing 2. The buttons 34 are manually depressible in a direction substantially perpendicular to the axis 80 of the cannister 5 which makes them easy to grip and move by a finger and thumb, as can be seen in FIG. 1. The buttons 34 load the loading member 32 and loading spring 31 through the arrangement illustrated in FIG. 10 comprising two torsion springs 35a and 35b fixed inside the upper housing portion. The torsion springs 35a and 35b engage the enlarged head 33 of the loading member 32 and respective ones of the buttons 34 to convert sideways force applied to the buttons 34 to a downwards force along the axis of the loading member shaft 32.

An alternative means for converting the sideways force applied to the buttons 34 is illustrated in FIG. 11. This consists of a double knee joint 36 fixed at its upper end 37 to the upper housing portion 3, fixed at its lower end 38 to the enlarged head 33 of the loading member 32 and fixed at its intermediate joints 39a and 39b to the respective buttons 34a and 34b.

The pre-loading mechanism further includes a lever 40 pivoted relative to the housing about a pivot 41. The lever 40 has a planar canister engagement portion 42 contacting the canister body 18 adjacent the pivot 41 with a pair of arms 43 and 44 extending therefrom. One arm 43 is engaged by the loading spring 31 so that the loading spring 31, when loaded, biasses compression of the canister through the lever 40 coupled to the canister 5 by the canister engagement portion 42. As the loading spring 31 is further away from the pivot 41 than the cannister engagement portion 42, this provides leverage between the loaded actuation force and the force applied to the cannister 5. The arm 43 has a hole through which the loading member shaft 32 extends. The other arm 44 of the lever 43 has a similar hole through which extends a further shaft 78 for preventing lateral displacement of the lever 40.

The actuation mechanism further includes a triggering mechanism for holding the lever 40 against compression of the canister under the biassing of the spring 31 and to release the lever 40 in response to inhalation at the mouthpiece. The triggering mechanism is constructed as follows.

The triggering mechanism comprises a first knee joint 45 having two links 46 and 47 connected pivotally to one another by a central pivot 50. The upper link 46 is pivotally connected both arms 43 and 44 of the lever 40 by a pivot 48. The lower link 47 is pivotally connected to the upper housing portion 3 by a pivot 49.

Accordingly, the first knee joint 45 has a locked position illustrated in FIGS. 7 to 9 in which it holds the lever 40 against compression of the canister 5. In the locked position of the first knee joint 45, the central pivot 50 is substantially aligned with the pivots 48 and 49 at the ends of the links 46 and 47. As the first knee joint 45 is connected to the lever at a position further away from the pivot 41 then the cannister engagement portion 42, this provides leverage between the locking force provided by the first knee joint and the force applied to the cannister 5. This leverage enhances the locking and triggering action of the triggering mechanism.

The triggering mechanism further includes a second knee joint 51 comprising two links 52 and 53 connected by a central pivot 54. One link 57 of the second knee joint 51 is pivotally connected to the upper housing portion 3 by a pivot 55 and extends laterally so that it constitutes a trigger vane which is moved by a flow of air thereover. The trigger vane 52 has a counterweight portion 79 (illustrated only in FIG. 7) fixed to the opposite side of pivot 55 from the laterally extending surface. The counterweight balances the trigger vane so that its centre of mass is positioned on the axis of the pivot 55.

The other link 53 of the second knee joint 51 extends from the trigger vane 52 between the arms 43, 44 of the lever 40 to the upper link 46 of the first knee joint 45 where it is pivotally connected by a pivot 56.

Accordingly, the second knee joint 51 has a locked position illustrated in FIGS. 7 to 9. In the locked position of the second knee joint, the central pivot 54 is substantially aligned with the pivots 55 and 56 and the ends of the links 52 and 53.

The actuation mechanism 6 further includes a reset mechanism which is constructed as follows.

The reset mechanism employs a locking element constituted by a third knee joint 57 comprising an upper link 58 and a lower Link 59 pivotally connected together by a central pivot 60. The upper link 58 is pivotally connected to the upper housing portion 3 by the pivot 49 in common with the first knee joint 45. The lower link 59 is pivotally connected to the loading-member shaft 32 by a pivot 61. The third knee joint 57 has a locked position illustrated in FIGS. 7 to 9 in which it holds the loading member shaft 32 in its loaded position as illustrated in FIG. 7. In the locked position of the third knee joint 57, the central pivot 60 is aligned with the pivots 48 and 61 at the end of the links 58 and 59. The third knee joint 57 is also biassed into its locked position by a biassing spring 67 connected to the upper housing portion 3. Hence the third knee joint constitutes a locking element which holds the canister in a compressed state through spring 31 and lever 40 after the full movement of the lever 40 to compress the canister 5.

The reset mechanism further includes a release member 62 mounted on the loading member shaft 32 by having an aperture through which the shaft 32 extends. The release member 62 is movable relative to the shaft 32 between limits defined by a pin 63 protruding from the shaft 32 engaging in a track 64 formed in the release member 62. A timer spring 65, the coils of which encircle the shaft 32, is connected between the arm 43 of the lever 40 and the release member 62. The timer spring 65 is in a relaxed state in FIG. 7 and is provided for biassing the release member 62 when loaded by movement of the lever 40 to compress the canister 5.

A protrusion 66 extends from the release member 62 (as best seen in the partial view of FIG. 12) to engage with the lower link 59 of the third knee joint 57 when the release member 62 is moved down the shaft 32. Such engagement of the protrusion 56 with the third knee joint 57 moves the knee joint 57 against the biassing spring 67 to break the third knee joint 57 thereby releasing locking effect of the third kneejoint 57.

The shaft 32 is biassed upwardly by a reset spring 68 acting between the shaft 32 and upper housing portion 3 to move the shaft 32 upwardly upon breaking of the third kneejoint 57.

The downwards movement of the release member 62 is damped by a damping element 69 consisting of a stator 70 fixed to the upper housing portion 3 and a rotor 71 rotatable through viscous fluid provided between the rotor 71 and stator 70. The rotor 71 is driven by a toothed rack 72 connected to the release member 62.

Operation of the actuation mechanism 6 will now be described with reference to FIGS. 13 to 16 which illustrate the various parts of the actuation mechanism 6 in schematic form for ease of understanding.

FIG. 13 illustrates the neutral state in which the loading member shaft 32 is in its uppermost position, so that the loading spring 31 is relaxed. In this state, the first and second knee joints 45 and 51 are both in their locked positions. The timer spring 65 and the reset spring 68 are relaxed.

Upon depression of the buttons 34, the loading member shaft 32 is moved downwardly to a second position illustrated in FIG. 14 loading the loading spring 31 which therefore biasses the lever 40 towards compression of the canister 5. However, the fist knee joint 45 is its locked position where it holds the lever 40 against compression of the canister 5. The first knee joint 45 is held in its own locked position by the second kneejoint 51 being in its locked position.

Movement of the loading member shaft 32 downwards also loads the reset spring 68 and brings the third knee joint 57 into its locked position where it is held by the spring 67. In this loaded state illustrated in FIG. 14, the inhaler 1 is loaded ready for delivery of a dose of medicament.

Inhalation by the user at the mouthpiece 13 generates an air flow through the air flow path defined inside the housing 2 from the inward opening 29 to the mouthpiece 13. This air flow acts on the trigger vane 55 of the second knee joint 51 causing it to move upwardly due to pressure drop created by the flow inside the housing 2 to the position illustrated in FIG. 15 where the second knee joint is broken. This breaks the first knee joint 45 into its broken position illustrated in FIG. 15 which releases the lever 40 and allows it to compress the canister 5 under the biassing of the loading spring 31.

During compression of the canisters, the shaft 32 remains locked in position by the third knee joint 37. This causes the canister to be held in its compressed state by the shaft 2 acting through the spring 31 and lever 40, the spring force of the spring 31 far exceeding the internal reset biassing of the canister 5.

However, movement of the lever 40 loads the timer spring 65 which accordingly biasses the release member 62 downwards. Movement of the release member 62 is delayed by the damping action of the damping element 69. The protrusion 66 of the release element 62 engages the third knee joint 57 after a predetermined period of time after actuation of the canister 5. This time is determined by the strength of the timer spring 65 and the damping properties of the damping element 69 and is at least 100 ms or 200 ms and up to 1000 ms or 5000 ms to allow the full dose of medicament to be delivered from the cannister 5. Such engagement breaks the third knee joint 57 into its broken position as illustrated in FIG. 16. Subsequently the reset spring 68 moves the loading member shaft 32 upwardly to the neutral position illustrated in FIG. 13. At the same time the shaft 32 lifts the release member 62, itself still damped by the damping element 69 so that the reset movement is damped.

Release of the shaft 32 causes the spring 31 to raise the lever 40 which has two effects. Firstly it allows the canister to reset itself. Secondly, it causes the first and second knee joints 45 and 51 to straighten, returning them to their locked position in the neutral position of the actuator mechanism illustrated in FIG. 13. The loading spring 31 and the timer spring 65 are pre-loaded and do not work against the reset movement, so that the reset spring 68 has only to overcome friction and the weight of the component.

The buttons 34a and 34b protrude from the inhaler when the actuation is in its relaxed state as shown in FIG. 1 and are depressed to a position flush with the side walks 8 of the upper housing portion 3. Accordingly, the distance between the extremities of the buttons before depression is less than the maximum length of the inhaler 1 in the direction parallel to the axis 80 of the canister 5 and less than the overall length of the canister 5 including the body 18 and the valve stem 19. Also, the total distance over which the two buttons 34 are moved relative to one another is greater than the distance by which the body 18 and the valve stem 19 of the canister 5 are relatively compressed. This is achieved by the leverage obtained by the loading spring 31 engaging lever 40 at a point further away from the pivot 41 than the canister engagement portion 42.

The actual flow recommended in order to correctly deliver a drug will depend on the manner operation of the drug the position where it should be deposited in the mouth, lungs of the user and the manner of dispensing the drug.

Some drugs are inhaled as a fine mist and transported all the way to the lungs whereas others are inhaled like a jet of liquid deposited in the mouth of the person.

These different types of drugs require different types of inhalation and therefore different inhalation flows and different actions by the user.

It is possible to adapt each of a number of different inhalers for use with a number of different types of drug by giving each inhaler a vent with a different shape and giving different closure elements shapes which conform with a single type of inhaler. For example, a possible different shape is illustrated by the dotted line in FIG. 1. Thus canister with differently shaped closure elements are for use exclusively with the inhaler having a conforming vent. The different shapes may prevent a closure element from being fitted in an inhaler of the inhaler having a conforming vent. Alternatively, the closure element may fail to close the vent of an inhaler having a differently shaped vent such that the remaining opening vents the flow sufficiently to prevent operation of the triggering mechanism.

FIG. 17 illustrates an electronic system for indicating to the user how long inhalation should be maintained or exhalation should be delayed.

A switch 73 is fixed to the inside of a side wall 7 of the upper housing portion 3 to be physically switched by movement of the lever 40 compressing the canister 5 The signal from the switch 73 is passed to a microprocessor 74 to indicate when the canister is actuated. At that point, the microprocessor 74 starts a timing count to measure the elapsed time from actuation of the canister.

When a predetermined elapsed time has elapsed, the microprocessor 74 sends an operation signal to operate three different indicator means, namely a display 75, a buzzer 76 and a vibrator 77.

The display 75 is arranged in the front wall 8 of the upper housing portion 3 as illustrated in FIG. 1. On receiving the operation signal from the microprocessor 74, the display is changed to provide a visual indication that the predetermined time has elapsed. The microprocessor 74 may also send control signals to the display 75 in response to actuation of the canister 5 detected by a switch 73 to cause the display 75 to display the number of doses remaining in the canister. In this case, the visual indication that the predetermined time has elapsed may be to alter the disclosed number of doses, for example, by blanking or flashing the displayed count.

The operation signal sent from the microprocessor 74 to the buzzer 76 causes it to generate a buzzing sound as an audible indication that the predetermined time has elapsed. Similarly, the signals sent from the processor 74 to the vibrator 77 causes the vibrator to shake the inhaler 1, for example by moving a mass within the vibrator 77.

Similarly, the microprocessor may be programmed to send an operation signal to operate the indicator means 75, 76 and 77 when a second predetermined time has elapsed from actuation of the canister.

The first and second elapsed times are selected to be timed during which the user should maintain inhalation or delay exhalation for proper delivery of a medicament in the canister 5, typically between 0.2 s and 15 s. In this way, the user is provided with an indication of how to breathe correctly which assists in ensuring a proper delivery of a dose

1. An inhaler for delivering medicament by inhalation, comprising:
a housing for receiving a canister of medicament actuatable to deliver a dose of medicament;
a detector for detecting actuation of a received canister;
a timer responsive to the detector to measure the elapsed time from actuation of the canister; and
an indicator means responsive to the timer to indicate when a predetermined time has elapsed during which the user should maintain inhalation or delay exhalation.

2. An inhaler according to claim 1, wherein the period of time is from 0.2 to 15 seconds.

3. A inhaler according to claim or 2, wherein the indicator means is a display for displaying a visual indication the predetermined time has elapsed.

4. An inhaler according to claim 3, wherein the display is arranged in normal use to display a representation of the number of doses remaining in the canister and to alter that representation when said predetermined time has elapsed.

5. An inhaler according to any one of claims 1 to 3, wherein the indicator means is arranged to generate an audible indication of the predetermined time has elapsed.

6. An inhaler according to any one of claims 1 to 3, wherein tile indicator means physically moves the inhaler.

7. An inhaler according to any one of the preceding claims, wherein the indicator means is further responsive to the timer to indicate when a second predetermined time has elapsed.

8. An inhaler according to claim 7, wherein the first mentioned predetermined time is a time during which the user should maintain inhalation and the second predetermined time as a time during which the user should delay exhalation.

9. An inhaler according to any one of the preceding claims, wherein the inhaler further comprising an actuation mechanism operatable to actuate the canister and the detector is arranged to detect operation of the actuation mechanism.

10. An inhaler according to claim 9, wherein the actuator mechanism is breath actuated.
